(19) United States
(12) Patent Application Publication
(10) Pub. No.: US 2012/0029018 A1
(43) Pub. Date: Feb. 2, 2012

(54) 9-SUBSTITUTED PHENANTHRENE BASED TYLOPHORINE DERIVATIVES

(76) Inventors: Kuo-Hsiung Lee, Chapel Hill, NC (US); Qian Shi, Chapel Hill, NC (US); Xiaoming Yang, Chapel Hill, NC (US); Kenneth F. Bastow, Chapel Hill, NC (US); Jau-Chen Lin, Taipei (TW); Pan-Chyr Yang, Taipei (TW)

(21) Appl. No.: 13/059,426

(22) PCT Filed: Aug. 25, 2009
(86) PCT No.: PCT/US09/04828

§ 371 Date: Mar. 29, 2011

Related U.S. Application Data
(60) Provisional application No. 61/091,806, filed on Aug. 26, 2008.

(51) Int. Cl. A61K 31/4525; A61P 35/00; A61P 35/02; C07D 405/10

(52) U.S. Cl. 514/321; 546/197

(57) ABSTRACT

The present invention provides compounds of Formula I: compositions containing the same, and methods of use thereof such as for the treatment of cancer.

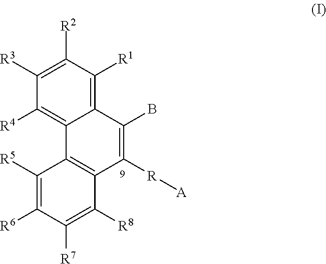

### STATEMENT OF GOVERNMENT SUPPORT

This invention was made with government support under NIH grant CA 17625. The Government has certain rights to this invention.

### FIELD OF THE INVENTION

The present invention concerns phenanthrine-based tylophorine (PBT) analogs as active compounds, formulations thereof, and methods of use thereof, particularly in methods of treating cancer.

### BACKGROUND OF THE INVENTION

The phenanthroindolizidine and phenanthroquinolizidine alkaloids are a class of pentacyclic natural products isolated primarily from species of Cynanchum, Pergularia, and Tylophora in the Asclepiadaceae family.^{1,2} The potent cytotoxic effect associated with tylophorine using antitumor screening launched by National Cancer Institute has aroused a great interest in exploring the synthesis and studying the structure and activity relationship of these compounds. The goal of these efforts is to obtain higher inhibitory potency and lower side effects, especially reduce or avoid the associated-CNS toxicity.³ Although the biochemical target of tylophorine is still unknown, recent research indicated that the NFκb signaling pathway and the synthesis of a number of cell cycle proteins such as cyclin D₁ were suppressed during the course of its action.^{4,5}

### SUMMARY OF THE INVENTION

A first aspect of the present invention is a compound of Formula I:

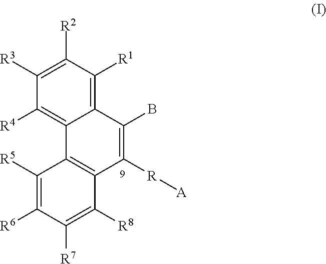

wherein:

R is C₁-C₄ alkylene;

A is as described below;

B is H, halo, loweralkyl, or loweralkenyl; and

R¹, R², R³, R⁴, R⁵, R⁶, R⁷ and R⁸ are each independently selected from the group consisting of H, halo, alkoxy, loweralkyl, and loweralkenyl;

or a pharmaceutically acceptable salt thereof.

In some embodiments, at least one of R², R³, R⁴, R⁵, R⁶ and R⁷ is alkoxy.

In some embodiments, (a) R² and R³ together form —O—CH(R¹⁰)—O—, or (b) R⁵ and R⁶ together form —O—CH(R¹⁰)—O—, wherein R¹⁰ is H, halo, or loweralkyl;

A further aspect of the present invention is a pharmaceutical formulation comprising an active compound as described herein, in a pharmaceutically acceptable carrier (e.g., an aqueous carrier).

A still further aspect of the present invention is a method of treating a cancer, comprising administering to a human or animal subject in need thereof a treatment effective amount (e.g., an amount effective to treat, slow the progression of, etc.) of an active compound as described herein. Examples of cancers that may be treated include, but are not limited to, skin cancer, lung cancer including small cell lung cancer and non-small cell lung cancer, testicular cancer, lymphoma, leukemia, Kaposi's sarcoma, esophageal cancer, stomach cancer, colon cancer, breast cancer, endometrial cancer, ovarian cancer, central nervous system cancer, liver cancer and prostate cancer.

A still further aspect of the invention is the use of an active compound or active agent as described herein for the preparation of a medicament for carrying out a method of treatment as described herein.

### BRIEF DESCRIPTION OF THE DRAWINGS

FIG. 1 shows that PBT-1 suppressed colony formation of lung cancer cells in vitro (photographs of cell plates not shown)

FIG. 2 shows that PBT-1 could also suppress Akt activation, and accelerate RelA (p65) degradation via IκB kinase-α, and downregulate the expressions of NF-κB target genes (FIG. 2; gel photographs not shown).

### DETAILED DESCRIPTION OF THE INVENTION

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs. All publications, patent applications, patents and other references mentioned herein are incorporated by reference in their entirety.

“Alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Loweralkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like.

“Alkenyl,” as used herein, refers to a straight or branched chain hydrocarbon containing from 2 to 10 carbons and containing at least one carbon-carbon double bond formed by the removal of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. “Loweralkenyl” as used herein, is a subset of alkenyl and refers to a straight or branched chain hydrocarbon group containing from 1 to 4 carbon atoms.

“Alkoxy,” as used herein, refers to an alkyl group, as defined herein, appended to the parent molecular moiety through an oxy group, as defined herein. Representative examples of alkoxy include, but are not limited to, methoxy, ethoxy, propoxy, 2-propoxy, butoxy, tert-butoxy, pentyloxy, hexyloxy and the like.

“Alkylthio” as used herein refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Cycloalkyl,” as used herein, refers to a saturated cyclic hydrocarbon group containing from 3 or 4 to 6 or 8 carbons. Representative examples of cycloalkyl include, cyclopropyl, cyclobutyl, cyclopentyl, cyclohexyl, cycloheptyl, and cyclooctyl.

“Aryl” as used herein, refers to a monocyclic carbocyclic ring system or a bicyclic carbocyclic fused ring system having one or more aromatic rings. Representative examples of aryl include, but are not limited to, azulenyl, indanyl, indenyl, naphthyl, phenyl, tetrahydronaphthyl, and the like. In some embodiments the aryl is a heterocycle as described below.

“Heterocycle,” as used herein, refers to a monocyclic- or a bicyclic-ring system. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, thiopyranopyridine, and the like.

Heterocycle groups of this invention can be substituted with 1, 2, or 3 substituents, such as substituents independently selected from alkenyl, alkenyloxy, alkoxy, alkoxyalkoxy, alkoxycarbonyl, alkyl, alkylcarbonyl, alkylcarbonyloxy, alkylsulfinyl, alkylsulfonyl, alkylthio, alkynyl, aryl, azido, arylalkoxy, arylalkoxycarbonyl, arylalkyl, aryloxy, carboxy, cyano, formyl, oxo, halo, haloalkyl, haloalkoxy, hydroxy, hydroxyalkyl, mercapto, nitro, sulfamyl, sulfo, sulfonate, —NR′ R″ (wherein, R′ and R″ are independently selected from hydrogen, alkyl, alkylcarbonyl, aryl, arylalkyl and formyl), and —C(O)NRR′ (wherein, R and R′ are independently selected from hydrogen, alkyl, aryl, and arylalkyl).

“Halo” as used herein refers to any halogen group, such as chloro, fluoro, bromo, or iodo.

“Oxo” as used herein, refers to a ═O moiety.

“Oxy,” as used herein, refers to a —O— moiety.

“Amine” or “amino group” is intended to mean the radical —NH₂.

“Substituted amino” or “substituted amine” refers to an amino group, wherein one or two of the hydrogens is replaced by a suitable substituent. Disubstituted amines may have substituents that are bridging, i.e., form a heterocyclic ring structure that includes the amine nitrogen as the linking atom to the parent compound. Examples of substituted amino include but are not limited to alkylamino, dialkylamino, and heterocyclo (where the heterocyclo is linked to the parent compound by a nitrogen atom in the heterocyclic ring or heterocyclic ring system).

“Alkylamino” is intended to mean the radical —NHR′, where R′ is alkyl.

“Dialkylamino” is intended to mean the radical NR′R″, where R′ R″ are each independently an alkyl group.

“Treat” or “treating” as used herein refers to any type of treatment that imparts a benefit to a patient afflicted with a disease, including improvement in the condition of the patient (e.g., in one or more symptoms), delay in the progression of the disease, prevention or delay of the onset of the disease, etc.

“Pharmaceutically acceptable” as used herein means that the compound or composition is suitable for administration to a subject to achieve the treatments described herein, without unduly deleterious side effects in light of the severity of the disease and necessity of the treatment.

“Inhibit” as used herein means that a potential effect is partially or completely eliminated.

The present invention is concerned primarily with the treatment of human subjects, but may also be employed for the treatment of other animal subjects (i.e., mammals such as dogs, cats, horses, etc. or avians) for veterinary purposes. Mammals are preferred, with humans being particularly preferred.

### A. Active Compounds.

Active compounds of the present invention are, in general, compounds of Formula I:

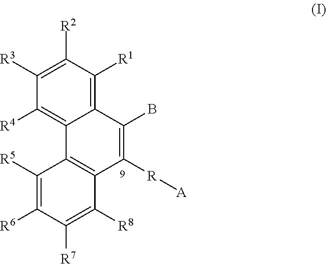

wherein:

R is C₁-C₄ alkylene (e.g., —CH₂—, —CH₂CH₂—, —CH₂CH₂CH₂—));

A is as described below;

B is H, halo, loweralkyl, or loweralkenyl;

R¹, R², R³, R⁴, R⁵, R⁶, R⁷ and R⁸ are each independently selected from the group consisting of H, halo, alkoxy, loweralkyl, and loweralkenyl;

subject to the proviso that at least one of R², R³, R⁴, R⁵, R⁶ and R⁷ is alkoxy;

and subject to the proviso that either (a) R² and R³ together form —O—CH(R¹⁰)—O— (as shown in Formula Ia below), or (b) R⁵ and R⁶ together form —O—CH(R¹⁰)—O— (as shown in Formula Ib below), wherein R¹⁰ is H, halo, or loweralkyl;

and pharmaceutically acceptable salts thereof.

More particular examples of compounds of Formula I include compounds of Formulas Ia and Ib:

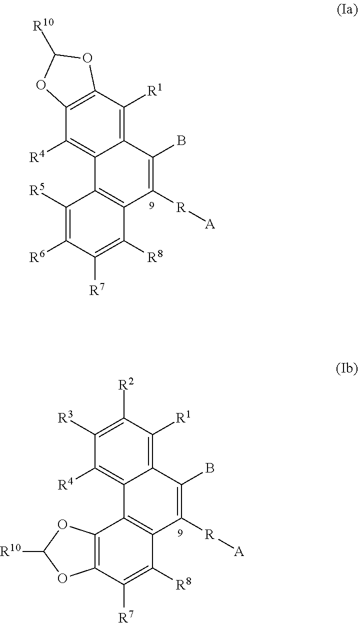

wherein A, B, R, R¹, R², R³, R⁴, R⁵, R⁶, R⁷, R⁸ and R¹⁰ are as given above or below.

Substituent “A” is selected from the group consisting of:

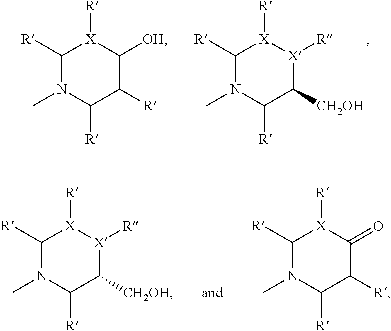

wherein X and X′ are each independently selected from N, O, and C, and each R′ and R″ is independently selected from the group consisting of H, alkyl, hydroxyalkyl, alkenyl, alkoxy, halo, oxo (═O), ═S, amino, substituted amino, alkoxyalkyl, alkylthiolkyl, and aryl (e.g., phenyl), all of which are optionally substituted (e.g., with hydroxyl, preferably at the para position) subject to the proviso that the corresponding R′ is absent when X is O or S. Particularly preferred examples of substituent “A” are:

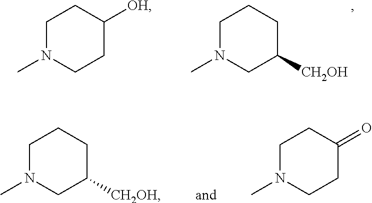

In some embodiments of the foregoing, R² and R³ are both alkoxy, such as methoxy or ethoxy.

In some embodiments of the foregoing, R³ is alkoxy, such as methoxy or ethoxy.

In some embodiments of the foregoing, R⁶ is alkoxy, such as methoxy or ethoxy.

In some embodiments of the foregoing, R⁵ and R⁶ are both alkoxy, such as methoxy or ethoxy.

In some embodiments of the foregoing, R⁶ and R⁷ are both alkoxy, such as methoxy or ethoxy.

Compounds of the present invention can be made in accordance with known techniques, such as the Perkin reaction (See, Wassmundt, F. W.; Kiesman, W. F., J. Org. Chem. 1995; 60:196-201; Lebrun, S et al., Tetrahedron 1999, 55, 2659-2670) and improved free-radical Pschorr cyclization (Gellert, E. In Alkaloids: Chemical and Biological Perspectives; Pelletier, S. W. Ed.; Academic Press: New York, 1987; pp 55-132.), or variations thereof which will be apparent to those skilled in the art based upon the disclosure provided herein.

### B. Formulations and Pharmaceutically Acceptable Salts.

The term “active agent” as used herein, includes the pharmaceutically acceptable salts of the compound. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine.

Active agents used to prepare compositions for the present invention may alternatively be in the form of a pharmaceutically acceptable free base of active agent. Because the free base of the compound is less soluble than the salt, free base compositions are employed to provide more sustained release of active agent to the target area. Active agent present in the target area which has not gone into solution is not available to induce a physiological response, but serves as a depot of bioavailable drug which gradually goes into solution.

The compounds of the present invention are useful as pharmaceutically active agents and may be utilized in bulk form. More preferably, however, these compounds are formulated into pharmaceutical formulations for administration. Any of a number of suitable pharmaceutical formulations may be utilized as a vehicle for the administration of the compounds of the present invention.

The compounds of the present invention may be formulated for administration for the treatment of a variety of conditions. In the manufacture of a pharmaceutical formulation according to the invention, the compounds of the present invention and the physiologically acceptable salts thereof, or the acid derivatives of either (hereinafter referred to as the “active compound”) are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.5% to 95% by weight of the active compound. One or more of each of the active compounds may be incorporated in the formulations of the invention, which may be prepared by any of the well-known techniques of pharmacy consisting essentially of admixing the components, optionally including one or more accessory ingredients.

The formulations of the invention include those suitable for oral, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above).

In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, or intradermal injection. Such preparations may conveniently be prepared by admixing the compound with water or a glycine buffer and rendering the resulting solution sterile and isotonic with the blood.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include vaseline, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3:318 (1986)) and typically take the form of an optionally buffered aqueous solution of the active compound. Suitable formulations comprise citrate or bis\tris buffer (pH 6) or ethanol/water and contain from 0.01 to 0.2M active ingredient.

### C. Methods of Use.

In addition to the compounds of the formulas described herein, the present invention also provides useful therapeutic methods. For example, the present invention provides a method of inducing cytotoxicity against tumor cells, or treating a cancer or tumor in a subject in need thereof.

Cancer cells which may be inhibited include cells from skin cancer, small cell lung cancer, non-small cell lung cancer, testicular cancer, lymphoma, leukemia, Kaposi's sarcoma, esophageal cancer, stomach cancer, colon cancer, breast cancer, endometrial cancer, ovarian cancer, central nervous system cancer, liver cancer and prostate cancer.

Subjects which may be treated using the methods of the present invention are typically human subjects although the methods of the present invention may be useful for veterinary purposes with other subjects, particularly mammalian subjects including, but not limited to, horses, cows, dogs, rabbits, fowl, sheep, and the like. As noted above, the present invention provides pharmaceutical formulations comprising the compounds of formulae described herein, or pharmaceutically acceptable salts thereof, in pharmaceutically acceptable carriers for any suitable route of administration, including but not limited to oral, rectal, topical, buccal, parenteral, intramuscular, intradermal, intravenous, and transdermal administration.

The therapeutically effective dosage of any specific compound will vary somewhat from compound to compound, patient to patient, and will depend upon the condition of the patient and the route of delivery. As a general proposition, a dosage from about 0.1 to about 50 mg/kg will have therapeutic efficacy, with still higher dosages potentially being employed for oral and/or aerosol administration. Toxicity concerns at the higher level may restrict intravenous dosages to a lower level such as up to about 10 mg/kg, all weights being calculated based upon the weight of the active base, including the cases where a salt is employed. Typically a dosage from about 0.5 mg/kg to about 5 mg/kg will be employed for intravenous or intramuscular administration. A dosage from about 10 mg/kg to about 50 mg/kg may be employed for oral administration.

The present invention is explained in greater detail in the following non-limiting examples.

### Example 1

In our previous research, we reported the finding and synthesis of a series of phenanthrene-based tylophorine (PBT) derivatives, in addition to some structure and activity relationship discussions regarding these PBTs. A variety of structural blocks were investigated including amino acid derivatives, pyrrolidine derivatives (substituted at C-2′), piperidines (substituted at C-2′ and C-4′), and piperazine derivatives. Of these compounds, compound 1 (PTB-1) was one of the most active compounds against four types of human cancer cell lines, including the multi-drug resistant (MDR) KB-VIN cells, with low IC₅₀ values at around 80 nM (Formula A).^{6,7.}

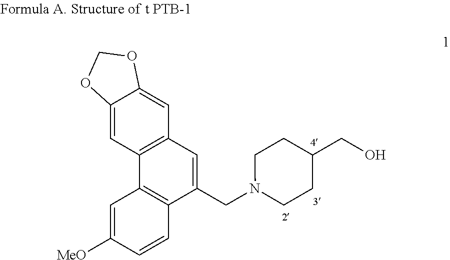

In view of these promising results obtained with a limited but diverse series of target compounds, further SAR study in order to explore the pharmacophore and to identify new potential drug leads using compound 1 as the new starting point appeared warranted. Therefore we synthesized a number of derivatives with different substituents at the C-3′ and C-4′ position of the piperidine ring. Amino groups were introduced to increase the water solubility and polarity of these compounds while retaining the ability to form hydrogen bond with the putative biochemical target. According to our earlier SAR work, a relative longer side chain in the piperidine ring led to a significant reduction in efficacy, we chose functional groups similar in size compared with the hydroxymethyl group of compound 1.

We also investigated substituents such as amino, aminomethyl, hydroxyl, hydroxylmethyl, methyl ester, cyano, trifluoromethyl, and methylsulfonylamino groups at C-3′ and C-4′ position. Using this established medicinal chemistry approach for ligand-based design, we hoped to obtain information useful in assisting further design and optimization.

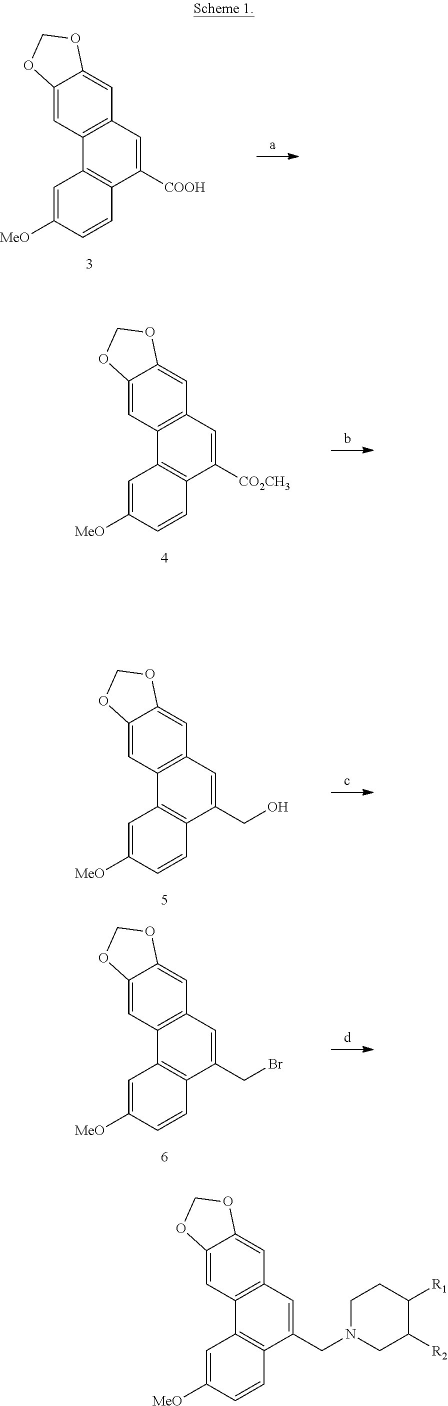

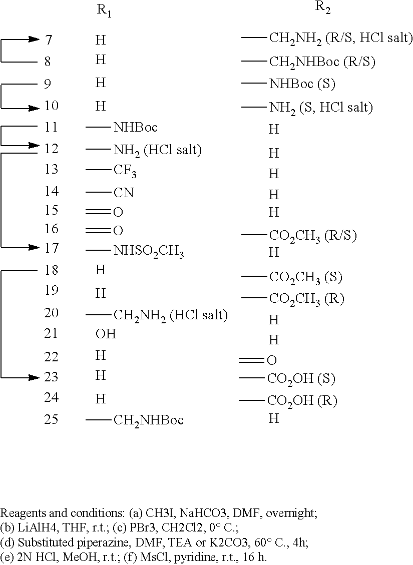

In Scheme 1, the general synthetic methods used to afford target derivatives are shown. The phenanthrene-9-carboxylic acid 3 obtained via 3 steps as reported in literature was reacted with methyl iodide using sodium bicarbonate to afford the methyl ester 4, which was then subject to LiAlH₄ reduction at room temperature to give the alcohol 5, followed by bromination using tribromophosphine in dichloromethane.⁸ For the final step, a variety of substituted piperidines were used to replace the bromine atom of 6 to afford our goal products at room temperature or 60° C. The Boc group was removed with HCl in MeOH and sulphonylamination was carried out in CH₂Cl_{2.9} Ketone was reduced with LiAlH₄ to form corresponding alcohol in excellent yields.

A total of 19 compounds were synthesized (two were R/S mixtures), and they were screened for in vitro anticancer activity against a panel of human tumor cell lines including KB (nasopharyngeal), A549 (lung), DU-145 (prostate), and KB-VIN (an MDR KB subline). The screening results are shown in Table 1. It can be concluded that most compounds exhibited significant activities, especially compounds 15 and 21, with IC₅₀ values in the ˜40 ng/ml range. Compound 21 was about 2-fold more active compared with compound 1, as indicated by lower IC₅₀ values (30-40 ng/ml). This augmented efficacy might be explained by a better match, especially a more suitable distance to form hydrogen bonds between the oxygen atom at C-4′ position and their potential targets (directly or indirectly), due to loss of one carbon atom in compound 1.

TABLE 1
In Vitro Anticancer Activity of Compounds 1 and 7-29.

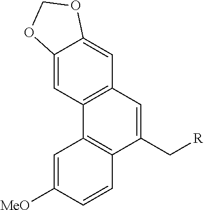

| | | IC₅₀ (μg/mL) | | | |
|---|---|---|---|---|---|
| Compounds | | KB | KBvin | A549 | DU145 |
| 7 | 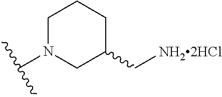 | 0.33 | 0.38 | 0.28 | 0.37 |
| 8 | 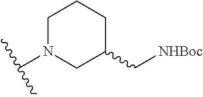 | 0.53 | 0.62 | 0.57 | 0.52 |
| 9 | 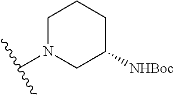 | 6.43 | 8.40 | 6.77 | 8.00 |
| 10 | 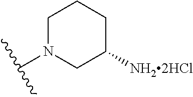 | 3.80 | 3.88 | 3.85 | 4.20 |
| 11 | 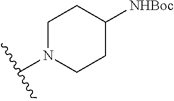 | 0.40 | 0.49 | 0.41 | 0.43 |
| 12 | 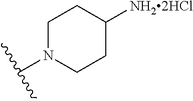 | 0.24 | 0.29 | 0.16 | 0.34 |
| 13 | 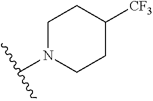 | 3.88 | 3.12 | 2.74 | 5.24 |
| 14 | 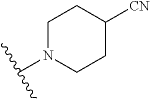 | 0.42 | 0.43 | 0.33 | 0.41 |
| 15 | 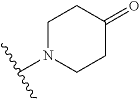 | 0.04 | 0.05 | 0.04 | 0.04 |
| 16 | 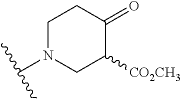 | 0.53 | 0.83 | 0.58 | 0.78 |
| 17 | 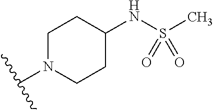 | 0.29 | 0.48 | 0.45 | 0.45 |
| 18 | 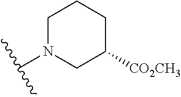 | 0.79 | 1.24 | 0.76 | 1.09 |
| 19 | 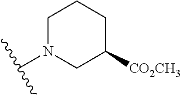 | 0.53 | 0.58 | 0.53 | 0.61 |
| 20 | 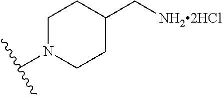 | 0.12 | — | 0.22 | 0.16 |
| 21 | 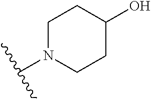 | 0.03 | — | 0.03 | 0.04 |
| 22 | 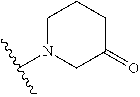 | 0.30 | — | 0.29 | 0.39 |
| 23 | 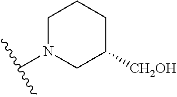 | 0.07 | — | 0.05 | 0.06 |
| 24 | 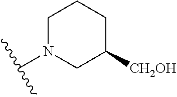 | 0.07 | — | 0.07 | 0.10 |
| 25 | 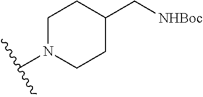 | 0.43 | — | 0.27 | 0.40 |
| 1 | 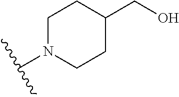 | 0.07 | 0.08 | 0.07 | 0.09 |

It is interesting to note that compound 15, the oxidized form of 21, possessed a similar high potency, indicating that the oxygen atom is primarily used as a hydrogen bond donor as long as the spatial distance was favorable, while a possible covalent adduct (ketone) is less likely to be formed. This was also evidenced by the fact that a longer side chain at C-3′ position (7, 8 vs. 9, 10) afforded a better profile of inhibition (closer to hydrogen bond acceptors). The relatively lower potency of 26 in comparison with 15 might arise from the steric resistance generated by the methyl ester group at the C-3′ position (R and S isomers), which might reduce the hydrogen bonding efficiency and affect its ideal conformations for binding. As for compound 16 vs. 18 and 19, the activities of the former were shown to be at the same level compared with the latters, again suggesting that the oxygen atom at C-4′ position might be expelled from its optimal hydrogen bonding angle as in 15, under which circumstances, the oxo group might not be involved in binding. When the oxo group was at the C-3′ position, the inhibitory potency decreased by about 8-fold, possibly induced by a potential interruption of the optimal hydrogen bonding in the pocket of the targets (15 vs. 22).

HCl salts showed a uniform increase in activities compared with their Boc protected precursors, probably resulting from elevated water solubility and side-chain shortening after removal of Boc, as demonstrated by 7, 10, 12, and 20. Lipophilic trifluoromethyl group substantially decreased their inhibitory activities as expected (compound 13). The cyano group (compound 14) resulted in a reduction in activity which might be associated with the oxidation state of nitrogen and special orientation of its lone electronic pair. For 11 and 17 vs. 12, a slight decrease of activities was observed, indicating that space might still be available for additional interaction in the pocket of corresponding target. After the methyl ester at C-3′ position was reduced to alcohol (23 and 24), the activities were significantly increased to the level of compound 1 and the potency difference induced by configuration at C-3′ was diminished. When the hydroxyl group was replaced by amino group, the activities greatly decreased, suggesting that formation of the hydrogen bond alone cannot interpret all the binding forces between these compounds and their targets.

In summary, we designed and synthesized 19 novel PBT-with different substitutions at C-3′ and C-4′ of the piperidine ring. A detailed SAR is reported and this data is used to interpret potential binding mechanisms for these PTB derivatives. Compounds 15 and 21 were identified as new more active PBT-1 derivatives.

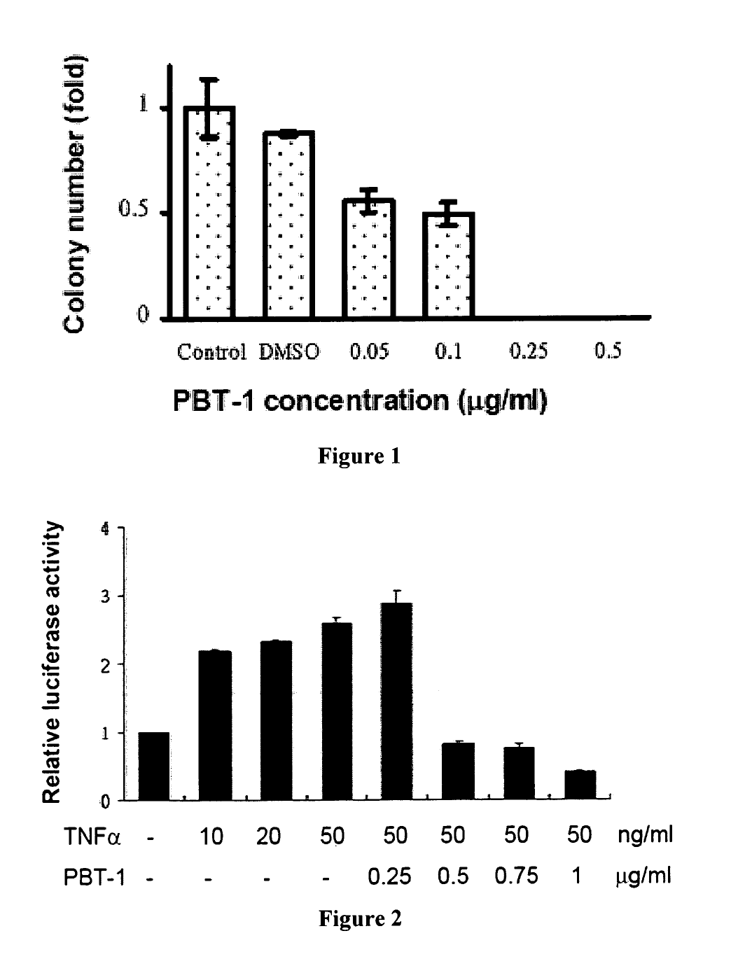

Tylophorine and related natural compounds exhibit potent antitumor activities. PBT-1, a synthetic C9-substituted phenanthrene-based tylophorine (PBT) derivative, significantly suppressed colony formation of lung cancer cells (FIG. 1; photos of in vitro culture plates omitted), and induced cell cycle G2/M arrest and apoptosis (Table 2). PBT-1 caused cyclin B1 and cyclin D1 protein accumulation in dose- and time-dependent manners. DNA microarray and pathway analysis showed that PBT-1 activated the apoptosis pathway and mitogen-activated protein kinase signaling. In contrast, PBT-1 suppressed the nuclear factor kappaB (NF-κB) pathway and focal adhesion. PBT-1 could also suppress Akt activation, and accelerate RelA (p65) degradation via IκB kinase-α, and downregulate the expressions of NF-κB target genes (FIG. 2; photos of gels omitted). The reciprocal recruitment of RelA and RelB on COX-2 promoter region led to the downregulation of transcriptional activity. In conclusion, PBT-1 may induce cell cycle G2/M arrest and apoptosis by inactivating Akt and inhibiting the NF-κB signaling pathways. PBT-1 may be a good drug candidate for anticancer chemotherapy.

TABLE 2

| CL1-0 | G1 (%) | S (%) | G2/M (%) |
|---|---|---|---|
| DMSO | 57.8 | 13.1 | 21.1 |
| 2 Hr. | 53.7 | 15.1 | 25.4 |
| 6 Hr. | 52.5 | 15.8 | 24.3 |
| 12 Hr. | 41.7 | 23.9 | 27.0 |
| 16 Hr. | 20.4 | 34.1 | 33.6 |
| 24 Hr. | 16.6 | 43.8 | 20.9 |

### REFERENCES

1. Baumgartner, B.; Erdelmeier, C. A. J.; Wright, A. D.; Rali, T.; Sticher, O. Phytochemistry 1990, 29, 3327-3330
2. Kim, S.; Lee, Y. M.; Lee, J.; Lee, T.; Fu, Y.' Song, Y. L.; Cho, J.; Kim D. J. Org. Chem. 2007, 72, 4886-4891
3. The 60-cell line test data of National Cancer Institute are accessible from the NSC numbers: http://dtp.nci.nih.gov/dtpstandard/dwindex/index.jsp.
4. Gao, W.; Lam, W.; Zhong, S.; Kaczmarek, C.; Baker, D. C.; Cheng, Y. C. Cancer Res 2004, 64, 678-688
5. Shiah, H. S.; Gao, W.; Baker, D. C.; Cheng, Y. C. Mol. Cancer. Ther. 2006, 5, 2484-2493
6. Wei, L.; Brossi, A.; Kendall, R.; Bastow, K. F.; Morris-Natschke, S. L.; Shi, Q.; Lee, K. H. Bioorganic & Medicinal Chemistry 2006, 14, 6560-6569
7. Wei, L.; Shi, Q.; Bastow, K. F.; Morris-Natschke, S. L.; Nakagawa-Goto, K.; Wu, T. S.; Pan, S. L.; Teng, C. M.; Lee, K. H. J. Med. Chem. 2007, 50, 3674-3680
8. Zhang, W.; Go, M. L. Euro. J. Med. Chem. 2007, 42, 841-850
9. Caldwell, J. J.; Davies, T. G.; Ruddle, R.; Raynaud, F. I.; Verdonk, M.; Workman, P.' Garrett, M. D.; Collins, I. J. Med. Chem. 2008, 51, 2147-2157

The foregoing is illustrative of the present invention, and is not to be construed as limiting thereof. The Invention is defined by the following claims, with equivalents of the claims to be included therein.

1. A compound of Formula I: 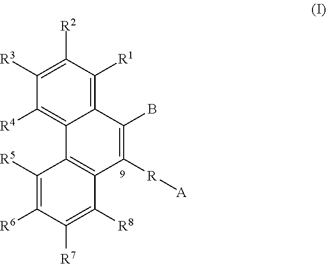
wherein:
R is C₁-C₄ alkylene;
B is H, halo, loweralkyl, or loweralkenyl;
R¹, R², R³, R⁴, R⁵, R⁶, R⁷ and R⁸ are each independently selected from the group consisting of H, halo, alkoxy, loweralkyl, and loweralkenyl;
subject to the proviso that at least one of R², R³, R⁴, R⁵, R⁶ and R⁷ is alkoxy;
and subject to the proviso that either (a) R² and R³ together form —O—CH(R¹⁰)—O—, or (b) R⁵ and R⁶ together form —O—CH(R¹⁰)—O—, wherein R¹⁰ is H, halo, or loweralkyl;
and wherein A is selected from the group consisting of: 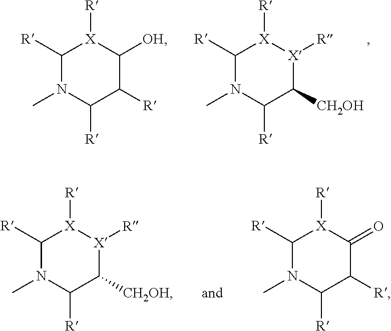
wherein X and X′ are each independently selected from N, O, and C, and each R′ and R″ is independently selected from the group consisting of H, alkyl, hydroxyalkyl, alkenyl, alkoxy, halo, oxo, ═S, amino, substituted amino, alkoxyalkyl, alkylthiolkyl, and aryl, subject to the proviso that the corresponding R′ or R″ is absent when X is O or S,
and pharmaceutically acceptable salts thereof.

2. A compound of claim 1 having Formula Ia: 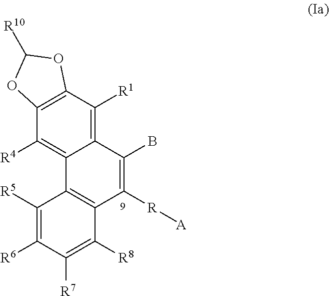
wherein A, B, R, R¹, R², R³, R⁴, R⁵, R⁶, R⁷, R⁸ and R¹⁰ are as given above;
and salts thereof.

3. A compound of claim 1 having Formula Ib: 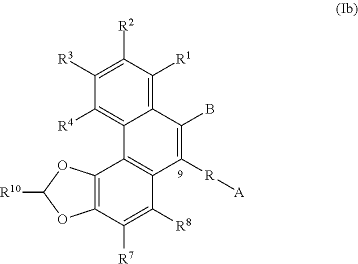
wherein A, B, R, R¹, R², R³, R⁴, R⁵, R⁶, R⁷ R⁸ and R¹⁰ are as given above;
and salts thereof.

4. The compound of claim 1, wherein each R′ is H.

5. The compound of claim 1, wherein A is selected from the group consisting of:

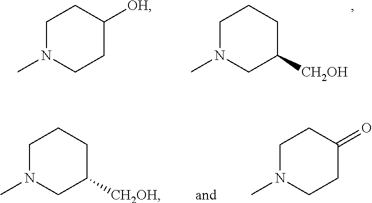

6. The compound of claim 5 having the structure of Formula Ic: 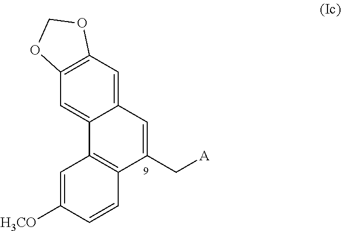
and wherein A is as given in claim 5.

7. A method of treating a cancer, comprising administering to a human or animal subject in need thereof a treatment effective amount of a compound of claim 1, or a pharmaceutically acceptable salt thereof.

8. The method of claim 7, wherein said cancer is selected from the group consisting of skin cancer, lung cancer, testicular cancer, lymphoma, leukemia, Kaposi's sarcoma, esophageal cancer, stomach cancer, colon cancer, breast cancer, endometrial cancer, ovarian cancer, liver cancer and prostate cancer.
sarcoma, esophageal cancer, stomach cancer, colon cancer, breast cancer, endometrial cancer, ovarian cancer, liver cancer and prostate cancer.

9. The method of claim 8, wherein said cancer is breast cancer.

10. The method of claim 8, wherein said cancer is lung cancer.

11. The method of claim 8, wherein said cancer is a multi-drug resistant cancer.

12. The method of claim 8, wherein said cancer is resistant to etoposide.

13-14. (canceled)
